(19) United States
(12) Patent Application Publication
(10) Pub. No.: US 2015/0361150 A1
(43) Pub. Date: Dec. 17, 2015

(54) METHODS AND COMPOSITIONS FOR TREATING GASTROINTESTINAL STROMAL TUMOR (GIST)

(71) Applicant: ROGER WILLIAMS MEDICAL CENTER, Providence, RI (US)

(72) Inventors: Steven C. Katz, Providence, RI (US); Richard P. Junghans, Boston, MA (US); Antony Bais, Providence, RI (US)

(21) Appl. No.: 14/763,787

(22) PCT Filed: Feb. 4, 2014
(86) PCT No.: PCT/US14/14635

§ 371 Date: Jul. 27, 2015

Related U.S. Application Data
(60) Provisional application No. 61/760,464, filed on Feb. 4, 2013.

(51) Int. Cl. C07K 14/52; A61K 38/17; A61K 45/06; A61K 38/19; C07K 14/725; C07K 14/705

(52) U.S. Cl. CPC C07K 14/52; C07K 14/7051; C07K 14/70521; A61K 45/06; A61K 38/19; A61K 38/1774; C07K 2319/74; C07K 2319/03

(57) ABSTRACT

The invention features nucleic acid constructs encoding chimeric immunoreceptors (CIRs) that are useful for treating a KIT+ associated disease in patients. In general, the CIRs contain an extracellular domain (e.g., a KIT-ligand (KL) or stem cell factor (SCF)) which interacts with and destroys KIT+ tumor cells, a transmembrane domain, and a cytoplasmic domain for mediating T cell activation (e.g., CD3 zeta and/or the domain of CD28). The invention also features the use of the nucleic acid constructs and/or host cells expressing CIRs in the treatment of a KIT+ associated disease, in particular gastrointestinal stromal tumor (GIST).

### CROSS-REFERENCE TO RELATED APPLICATION

This application claims benefit of priority to U.S. Provisional Application No. 61/760,464, filed Feb. 4, 2013, which is hereby incorporated by reference.

### BACKGROUND OF THE INVENTION

Gastrointestinal stromal tumor (GIST) is the most common GI mesenchymal neoplasm. Imatinib mesylate has been demonstrated to significantly prolong disease-free survival in the adjuvant setting and for patients with disseminated GIST. Unfortunately, the majority of patients with metastatic GIST who are treated with imatinib develop resistance and subsequently progressive disease. Therapeutic options are limited for patients who develop advanced GIST unresponsive to tyrosine kinase inhibitor (TKI) therapies such as imatinib. Consequently, there exists a need in the art for alternative therapies for treating GIST, particularly forms that are resistant to conventional therapies.

### SUMMARY OF THE INVENTION

The invention features a nucleic acid construct encoding a chimeric immune receptor (CIR) protein including: a) an extracellular domain of KIT-ligand (KL), or a fragment thereof, or a moiety specific for inhibiting dimerization of KIT (e.g., an antibody or antibody fragment), b) a transmembrane domain, and c) a cytoplasmic domain, wherein the transmembrane domain includes a domain of the CD3 zeta chain, or a fragment thereof, and the cytoplasmic domain includes a domain of the CD3 zeta chain, or a fragment thereof. The invention also features a nucleic acid construct encoding a CIR protein including: a) an extracellular domain of KIT-ligand, or a fragment thereof, or a moiety specific for inhibiting dimerization of KIT (e.g., an antibody or antibody fragment), b) a transmembrane domain, and c) a cytoplasmic domain, wherein the transmembrane domain includes a domain of CD28, or a fragment thereof, and the cytoplasmic domain includes a domain of the CD3 zeta chain and a domain of CD28, or fragments thereof.

In certain embodiments, the extracellular domain further includes a domain of CD28, or a fragment thereof. In one embodiment, the CIR protein, when expressed in a T cell, is capable of activating the T cell in the presence of a tyrosine-protein kinase KIT positive (KIT+) tumor cell. In another embodiment the extracellular domain of the CIR protein is capable of interacting with KIT on the surface of a tumor cell when expressed in a T cell.

The invention also features a vector including the nucleic acid constructs described above and a host cell including the nucleic acid constructs and vector. In one aspect, the host cell is selected from the group consisting of: a T cell, a hematopoietic stem cell, a natural killer cell, a natural killer T cell, a B cell, and a cell of monocytic lineage. In certain aspects, the host cell is autologous to the subject. In other aspects, the host cell is not autologous to the subject.

The invention features a method of destroying a KIT+ cell, the method including administering a composition including the nucleic acid construct described above. In some embodiments, the method includes contacting the KIT+ cell with a composition including the host cell as described above. The invention also features a method of treating a subject with a KIT+ associated disease, the method including administering a composition including a nucleic acid construct or a host cell described above.

Certain aspects of the invention include the administration of a second agent for treating a subject with a KIT+ associated disease or destroying a KIT+ cell. In certain embodiments, the second agent is a tyrosine-kinase inhibitor. In preferred embodiments, the tyrosine-kinase inhibitor is imatinib.

In all embodiments of the invention, the KIT+ associated disease is characterized by the presence of KIT+ tumor cells. In some embodiments, the KIT+ associated disease is selected from the group consisting of: gastrointestinal stromal tumor, acute myelogenous leukemia, small-cell lung carcinoma, ovarian carcinoma, breast carcinoma, melanoma, neuroblastoma, and soft-tissue sarcomas of neuroectodermal origin. In a preferred embodiment, the KIT+ associated disease is gastrointestinal stromal tumor (GIST). In other embodiments, the GIST is resistant to imatinib mesylate.

### DEFINITIONS

By “capable of interacting with KIT” is meant an extracellular domain which recognizes and binds tyrosine-protein kinase KIT, but does not substantially recognize and bind other molecules in a sample, e.g., a human blood sample.

By “treating” is meant ameliorating a condition or symptom(s) of a condition (e.g., the symptoms of gastrointestinal stromal tumor, acute myelogenous leukemia, small-cell lung carcinoma, ovarian carcinoma, breast carcinoma, melanoma, neuroblastoma, and soft-tissue sarcomas of neuroectodermal origin). To “treat a KIT+ associated disease” (e.g., gastrointestinal stromal tumor, acute myelogenous leukemia, small-cell lung carcinoma, ovarian carcinoma, breast carcinoma, melanoma, neuroblastoma, myelodysplastic syndrome (MDS), myeloproliferative disease (MPD), aggressive systemic mastocytosis (ASM), hypereosinophilic syndrome (HES), dermatofibrosarcoma protuberans (DFSP), soft-tissue sarcomas of neuroectodermal origin, heptocullular carcinoma, and all neoplasms derived from KIT+ stem cells) refers to administering a treatment to a subject with a KIT+ associated disease to improve the subject's condition. As compared with an equivalent untreated control, such amelioration or degree of treatment is at least 5%, 10%, 20%, 30%, 40%, 50%, 60%, 70%, 80%, 90%, 95%, 99%, or 100% improvement of the diseased-state.

By “vector” is meant a DNA molecule, usually derived from a plasmid or bacteriophage, into which fragments of DNA may be inserted or cloned. A recombinant vector will contain one or more unique restriction sites, and may be capable of autonomous replication in a defined host or vehicle organism such that the cloned sequence is reproducible. A vector contains a promoter operably-linked to a gene or coding region such that, upon transfection into a recipient cell, an RNA, or an encoded protein is expressed.

By “host cell” is meant a cell into which one or more nucleic acid constructs is introduced. Host cells can be isolated from a subject and/or isolated from an outside donor. Examples of host cell include but are not limited to T cells (e.g., isolated from human peripheral blood mononuclear cell, bone marrow, and/or thymus), hematopoietic stem cells, natural killer cells, natural killer T cells, B cells, and cells of monocytic lineage (e.g., blood monocytes and macrophages).

By “chimeric immunoreceptor” or “CIR” is meant a fusion protein which, when expressed in a host cell, contains an extracellular domain that specifically binds to a target protein and a cytoplasmic domain that modulates activation of the host cell.

By “KIT-ligand” is meant a cytokine that binds to tyrosine-protein kinase KIT (KIT), c-KIT receptor, or CD117. KIT-ligand binding to KIT can cause KIT to form a dimer that activates the intrinsic tyrosine kinase activity of the protein. KIT-ligand is also known in the art as steel factor or stem cell factor (SCF). “KIT-ligand” includes a polypeptide having at least residues corresponding to 1-273 of human KIT-ligand (SEQ ID NO:1) or e.g., a sequence having substantial identity to that of the extracellular domain of cKIT ligand (e.g., encoded by SEQ ID NOS:2,3 (italicized nucleotides indicate NcoI and BamHI restriction sites, respectively)).

```
Human KIT-ligand amino acid sequence
SEQ ID NO: 1
LOCUS AAI26167 273 aa linear PRI 23-OCT-2006
DEFINITION KIT ligand [Homo sapiens].
ACCESSION AAI26167
VERSION AAI26167.1 GI: 116496627
DBSOURCE accession BC126166.1
    1 mkktqtwilt ciylqlllfn plvktegicr nrvtnnvkdv tklvanlpkd ymitlkyvpg
  61 mdvlpshcwi semvvqlsds ltdlldkfsn iseglsnysi idklvnivdd lvecvkenss
121 kdlkksfksp eprlftpeef frifnrsida fkdfvvaset sdcvvsstls pekdsrvsvt
181 kpfmlppvaa sslrndssss nrkaknppgd sslhwaamal palfsliigf afgalywkkr
241 qpsltraven iqineednei smlqekeref qev
(5′-gattccaggaattgatttccccatggcaaagaagacacaaacttg-3′ SEQ ID NO: 2
5′-ctaagctctagccaattgaattggatccgtgtaggctggagtctcc-3′ SEQ ID NO: 3)
```

By “CD28 cytoplasmic domain” is meant a polypeptide having the C-terminal region of CD28 that is located in the cytoplasm when expressed in a T cell, e.g., a polypeptide having the amino acid sequence of amino acids 127-234 of SEQ ID NO:4. The term “CD28 cytoplasmic domain” is meant to include any CD28 fragment that maintains the ability to modulate activation of T cells and is substantially identical to amino acids 127-234 of SEQ ID NO:4 over the length of the polypeptide fragment.

```
Human CD28 amino acid sequence
SEQ ID NO: 4
LOCUS NP_006130 220 aa linear PRI 11-APR-2010
DEFINITION T cell-specific surface glycoprotein CD28 precursor [Homo sapiens].
ACCESSION NP_006130
VERSION NP_006130.1 GI: 5453611
DBSOURCE REFSEQ: accession NM_006139.2
    1 MLRLLLALNL FPSIQVTGNK ILVKQSPMLV AYDNAVNLSC KYSYNLFSRE FRASLHKGLD
  61 SAVEVCVVYG NYSQQLQVYS KTGFNCDGKL GNESVTFYLQ NLYVNQTDIY FCKIEVMYPP
  21 PYLDNEKSNG TIIHVKGKHL CPSPLFPGPS KPFWVLVVVG GVLACYSLLV TVAFIIFWVR
181 SKRSRLLHSD YMNMTPRRPG PTRKHYQPYA PPRDFAAYRS
```

By “CD3 zeta” is meant a polypeptide having polypeptide having the amino acid sequence of SEQ ID NO:5. The term “CD3 zeta” also includes polypeptide fragments that maintain the ability to modulate activation of T cells and is substantially identical to SEQ ID NO:5 over the length of the protein fragment.

```
Human CD3 zeta amino acid sequence
SEQ ID NO: 5
LOCUS NP_000725 163 aa linear PRI 11-APR-2010
DEFINITION T cell receptor zeta chain isoform 2 precursor [Homo sapiens].
ACCESSION NP_000725
VERSION NP_000725.1 GI: 4557431
DBSOURCE REFSEQ: accession NM_000734.3
    1 MKWKALFTAA ILQAQLPITE AQSFGLLDPK LCYLLDGILF IYGVILTALF LRVKFSRSAD
  61 APAYQQGQNQ LYNELNLGRR EEYDVLDKRR GRDPEMGGKP RRKNPQEGLY NELQKDKMAE
121 AYSEIGMKGE RRRGKGHDGL YQGLSTATKD TYDALHMQAL PPR
```

By “activating a T cell” is meant inducing a T cell expressing the CIR of the invention to release interleukin 2 (IL-2) which acts upon the T cell in an autocrine fashion. The activated T cells also produce the alpha sub-unit of the IL-2 receptor (CD25 or IL-2R), enabling a fully functional receptor that can bind with IL-2, which in turn activates the T cell's proliferation pathways.

By the term “tumor cell” is meant a component of a cell population characterized by inappropriate accumulation in a tissue. This inappropriate accumulation may be the result of a genetic or epigenetic variation that occurs in one or more cells of the cell population. This genetic or epigenetic variation causes the cells of the cell population to grow faster, die slower, or differentiate slower than the surrounding, normal tissue. The term “tumor cell” as used herein also encompasses cells that support the growth or survival of a malignant cell. Such supporting cells may include fibroblasts, vascular or lymphatic endothelial cells, inflammatory cells or co-expanded non-neoplastic cells that favor the growth or survival of the malignant cell. The term “tumor cell” is meant to include cancers of hematopoietic, epithelial, endothelial, or solid tissue origin. The term “tumor cell” is also meant to include cancer stem cells

By “KIT+ tumor cell” is meant a cell expressing KIT associated with a tumor.

By “KIT+ associated disease” is meant a disease that is characterized by increased KIT expression or aberrant KIT activity (e.g., KIT activation) in a variety of cell types, including but not limited to: mast cells, hematopoietic progenitor cells, melanocytes, germ cells, and/or gastrointestinal pacemaker cells. A KIT+ associated disease can arise from KIT+ stem cells. Examples of KIT+ associated diseases include but are not limited to: gastrointestinal stromal tumor, acute myelogenous leukemia, small-cell lung carcinoma, ovarian carcinoma, breast carcinoma, melanoma, neuroblastoma, myelodysplastic syndrome (MDS), myeloproliferative disease (MPD), aggressive systemic mastocytosis (ASM), hypereosinophilic syndrome (HES), dermatofibrosarcoma protuberans (DFSP), soft-tissue sarcomas of neuroectodermal origin, heptocullular carcinoma, and all neoplasms derived from KIT+ stem cells.

By “substantially identical” is meant a nucleic acid or amino acid sequence that, when optimally aligned, for example using the methods described below, share at least 60%, 65%, 70%, 75%, 80%, 85%, 90%, 95%, 96%, 97%, 98%, 99%, or 100% sequence identity with a second nucleic acid or amino acid sequence. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the full length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “subject” is meant a mammal (e.g., a human or non-human).

Other features and advantages of the invention will be apparent from the following Detailed Description, the drawings, and the claims.

### BRIEF DESCRIPTION OF THE DRAWINGS

FIGS. 1A-1B are depictions of the structure of anti-KIT chimeric immune receptor. FIG. 1A shows a schematic diagram of 1^{st} and 2^{nd} generation CIR genetic constructs. FIG. 1B shows the structure of 1^{st} and 2^{nd} generation CIR. Anti-KIT CIRs were re-engineered from anti-CEA retroviral vector constructs, whereby the extracellular domain of KIT ligand (KL) was amplified and cloned to replace the anti-CEA extracellular domain.

FIGS. 2A-2B are graphs showing the transduction efficiency and phenotype of human anti-KIT designer T cells. FIG. 2A shows a graph of isolated PBMC and primary human T cells activated and transduced with retrovirus expressing KIT-specific CIRs. Shaded histograms represent designer T cells and open curves represent untransduced cells. FIG. 2B shows flow cytometric analysis of the phenotype of 1^{st} and 2^{nd} generation transduced designer T cells demonstrating that T cells of a central memory phenotype (CD45RO+CD62LCCR7+) were in the majority. Data are representative of three or more repetitions.

FIGS. 3A-3G are graphs showing that human anti-KIT designer T cells retain proliferative ability in vitro. 1^{st} and 2^{nd} gen designer T cells were co-cultured with human KIT+GIST cell lines, GIST 882 in FIGS. 3A-3B, and 3E and GIST 48 in FIGS. 3C-3D, and 3F. Designer T cells and untransduced CTRL T cells were stained with CFSE and proliferation assessed by gating on CD3+ cells to measure CFSE dilution. To confirm designer T cells activated by KIT+GIST cell lines, IFNγ production was measured by ELISA, and was found to be in the range of 462-475 pg/mL, while that of unmodified T cells (CTRL) was negligible (G). Data are representative of three or more repetitions.

FIGS. 4A-4E are graphs showing the ability of human anti-KIT designer T cells to effectively lyse KIT+ tumor cells. FIG. 4A shows results from a LDH assay to evaluate enzymatic release following tumor cell lysis performed on supernatant from co-culturing of 1^{st} and 2^{nd} generation designer T cells with irradiated GIST 882 cells. Maximal release was defined by the highest experiment values. FIGS. 4B-4C show graphs of irradiated GIST-88s or GIST 48 cells labeled with CFSE and cultured with unlabelled T cells to confirm the cytotoxic ability of designer T cells. Tumor cell death was quantified by measuring the decrease in CFSE fluorescence by gating on remaining live cells. FIGS. 4D-4E show quantified results of the percent kill of 1^{st} and 2^{nd} gen designer T cells by normalizing MFI data to that of the unmodified cells (CTRL). Data are representative of at least three repetitions.

FIGS. 5A-5E are results of in vivo testing of human anti-KIT designer T cells. FIG. 5A show median results from a single representative's experiment of subcutaneous xenograft model whereby GIST 882 cells were injected subcutaneously into immunodeficient mice prior to treatment with human anti-KIT designer T cells and FIG. 5B shows results from pooled data from 3 experiments. FIG. 5C shows plots of the median tumor sizes at Day 5, Day 10, and Day 15 post-infusion indicated by horizontal bar along with individual tumor measurements represented by individual data points. FIG. 5D shows immunohistochemistry blots (top row 10x and bottom row 40x) detecting tumor infiltrating CD3+T cells (arrows). FIG. 5E shows routine histology blots used to assess the degree of tumor necrosis (asterisks) in mice treated with 1^{st} or 2^{nd} gen designer T cells, both with and without supplemental IL2 (left column 10x and right column 40x).

### DETAILED DESCRIPTION OF THE INVENTION

In general, the invention feature nucleic acid constructs encoding chimeric immune receptor (CIR) proteins that include KIT-ligand (KL) or stem cell factor (SCF). These nucleic acid constructs, when expressed in cells (e.g., a subject's T cells), are useful for the treatment of diseases associated with increased KIT expression or aberrant KIT activity. Examples of such diseases include gastrointestinal stromal tumors (GIST), particularly those resistant to imatinib and other conventional therapies. Certain embodiments include an engineered CIR that contains the natural ligand for KIT (e.g., KIT-ligand (KL) or stem cell factor (SCF). For example, KIT-ligand (KL) or stem cell factor (SCF) can be fused to the CD3ζ chain component of the T cell receptor (1^{st} generation, 1^{st} gen) or CD3ζ+the CD28 co-stimulatory molecule (2^{nd} generation, 2^{nd} gen). The 2^{nd} gen T cells express the construct that targets KIT+ tumors while, at the same time, integrating CD28 co-stimulatory signals. In the examples set forth below, such 1^{st} and 2^{nd} gen T cells were produced and tested in vitro and in vivo to demonstrate their efficacy in destroying KIT+ tumor cells.

### Extracellular Domains

The CIR of the invention feature an extracellular domain able to specifically bind tyrosine protein kinase KIT (KIT) and to direct the CIR of the invention to KIT+ expressing tumor cells. Therefore, the extracellular domain of the CIR of the invention can include, e.g., the full-length stem cell factor (SCF) or the KIT-ligand (KL), or fragments thereof. Alternatively, the extracellular domain can include any binding moiety specific for inhibiting dimerization of KIT, inhibiting tyrosine kinase activity, and/or KIT phosphorylation, and/or antibody fragments against KIT (e.g., an anti-cKIT antibody for example those commercially available from Abcam, Biolegend, Genway Biotech, etc).

The extracellular domain can optionally include a further protein tag useful for purification of the expressed CIR, e.g., a c-myc tag (EQKLISEEDL) of human origin, at the N-terminus, a Z domain, HA tag, FLAG tag, and/or GST tag. The protein tag is preferably chosen such that the tag does not obstruct KL interaction with KIT. The extracellular domain can also optionally include a further protein useful for imaging, e.g., fluorescent proteins. Inclusion of these proteins can facilitate future study of the constructs and to create constructs that are more effective.

### Transmembrane Domains

The CIR of the invention features transmembrane domains, e.g., those derived from CD28 or CD3 zeta. The inclusion of the transmembrane region of the zeta chain or the transmembrane and partial extracellular domain of CD28 provides the capability of intermolecular disulfide bonds. CIRs containing these transmembrane domains are predicted to form disulfide-linked dimers through a cysteine residue located in the transmembrane of zeta or in the proximal cysteine residue located in the partial extracellular domain of CD28 (position 123 of CD28), mimicking the dimer configuration of native zeta and CD28.

### Cytoplasmic Domains

The CIR of the invention also feature a cytoplasmic domain for modulating activation of the host T cells when bound to KIT+ tumor cells and to activate T cell based cytotoxic responses to attack the KIT+ tumor cells. In certain embodiments, cytoplasmic domains for use in the CIRs of the invention include CD3 zeta, CD28, or fragments thereof. The invention also features the fusion of polypeptides derived from multiple extracellular domains for potentiating activation of T cells when bound to KIT+ tumor cells (e.g., a cytoplasmic domain that includes both active fragments of CD3 zeta and CD28 or a cytoplasmic domain that includes only CD3 zeta).

### Nucleic Acid Constructs

The nucleic acid constructs of the invention are useful for expressing CIR constructs in host cells (e.g., T cells isolated from a subject). CIR constructs can be included in a single nucleic acid construct or multiple nucleic acid constructs. Nucleic acid sequences encoding the CIR can be changed to codons more compatible with an expression vector or host cell used for producing the CIR. Codons may be substituted to eliminate restriction sites or to include silent restriction sites, which may aid in cloning of the nucleic acid in an expression vector and processing of the nucleic acid in the selected host cell. In order to facilitate transfection of host cells, the nucleic acid construct can be included in a viral vector (e.g., a retroviral vector or adenoviral vector) or be designed to be transfected into a host cell via electroporation or chemical means (e.g., using a lipid transfection reagent).

### Host Cells

The host cells of the invention can be derived from any mammalian source containing T cells (e.g., human peripheral blood mononuclear cell, bone marrow, and/or thymus, and isolated from, e.g., a subject's own cells or from another donor source), hematopoietic stem cells, natural killer cells, natural killer T cells, B cells, and cells of monocytic lineage (e.g., blood monocytes and macrophages). The host cells are transfected or infected with the nucleic acid constructs of the invention (e.g., nucleic acid constructs encoding a CIR). Prior to administration to a patient, the host cell can be expanded in cell culture. In one embodiment, the modified host cells are administered to the patient from whom they were originally isolated. In another embodiment, the modified host cells are administered to a patient from another source from which they are isolated.

### Conditions and Disorders

Histologic studies confirm that the CIR of the invention localize to KIT+ tumors and mediate tumor necrosis after intravenous infusion. In some embodiments, single or multiple infusions may be necessary to achieve complete regression of established tumors. In particular embodiments, addition of IL15 and IL21 to promote an effector phenotype may offer the potential of enhancing the efficacy of the CIR of the invention. In specific embodiments, significant delays in tumor growth in the in vivo model support the potential utility of the CIR of the invention for the treatment of GIST. In another embodiment, the CIR of the invention may also be useful for the treatment of KIT+ associated diseases. Examples of KIT+ associated diseases include but are not limited to: acute myelogenous leukemia, small-cell lung carcinoma, ovarian carcinoma, breast carcinoma, melanoma, neuroblastoma, myeloproliferative disease (MPD), aggressive systemic mastocytosis (ASM), hypereosinophilic syndrome (HES), dermatofibrosarcoma protuberans (DSP), soft-tissue sarcomas of neuroectodermal origin, and/or any other disease characterized by the overexpression and/or hyper-activation of KIT.

### Additional Agents and Combination Therapy

The CIRs of the invention can are useful for GIST treatment and treatment of other KIT+ associated diseases that can be used alone or in combination with other therapies, including tyrosine kinase inhibitors (TKIs) and other immunomodulatory agents and/or therapy. Imatinib has been reported to induce regulatory T cell apoptosis and its efficacy was enhanced by concurrent immunotherapy. Adding imatinib to anti-KIT designer T cells infusions may augment efficacy through favorable immunomodulation within the tumor microenvironment, allowing the CIR to mediate enhanced tumor cell lysis. Other TKIs that can be used with the CIR of the invention include, but are not limited to, erlotinib, gefitinib, lapatinib, sunitinib, sorafenib, nilotinib, bosutinib, neratinib, and vatalanib. The CIRs of the invention can also be co-administered with immunomodulatory agents such as anti-PD1 and anti-CTLA4 antibodies. In any of the treatment methods of the invention, the CIRs of the invention can also be co-administered with myeloablative preconditioning as described in Dudley et al., J Clin Oncol. 26:5233-5239, 2008

### EXAMPLES

An anti-KIT CIR was constructed that can be utilized to reprogram human T cells to recognize and kill KIT+GIST tumors. The natural ligand for KIT, SCF, confers anti-tumor specificity. Murine and human T cells were transduced with high levels of efficiency, and in vitro studies confirmed that anti-KIT designer T cells proliferated and secreted IFNγ in response to KIT+GIST cells. The designer T cells were also able to lyse two KIT+ cell lines. Using a xenograft model, it was demonstrated that systemic infusions of 1^{st} and 2^{nd} gen anti-KIT designer T cells resulted in significant reductions in tumor growth rates. Taken together, the study of anti-KIT designer T cells supports their further development as a potential novel treatment for KIT+ neoplasms.

### Materials and Methods

### Retroviral Vector Construction

First and second generation anti-KIT CIR were re-engineered from the anti-CEA retroviral vector expression constructs previously described in Emtage et al., Clin Cancer Res 14:8112-8122, 2008. The extracellular domain of cKIT ligand (Genebank BC069733.1, cDNA clone MGC:97379) spanning the N-terminal start codon to the transmembrane start was PCR amplified from ATTC clone 010560371 using primers incorporating NcoI (italicized nucleotides in SEQ ID NO:2) and BamHI (italicized nucleotides in SEQ ID NO:3) restriction sites and cloned in-frame to replace the anti-CEA extracellular domain.

Nucleic acid encoding extracellular domain of cKIT ligand:

(5′-gattccaggaattgatttccccatggcaaagaagacacaaacttg-3′ SEQ ID NO: 2
5′-ctaagctctagccaattgaattggatccgtgtaggctggagtctcc-3′ SEQ ID NO: 3)

### Designer T Cell Production

Human peripheral blood mononuclear cells (PBMC) were obtained from random donor whole blood filtrate (Rhode Island Blood Center, Providence, R.I.). Blood filters were washed with sterile PBS (Cellgro, Manassas, Va.) and PBMC were isolated by density gradient separation with Histopaque (Sigma-Aldrich, St. Louis, Mo.) according to manufacturer directions. PBMC were seeded at a density of 2×10⁶ cells/ml, and activated on anti-CD3 coated (OKT3, eBioscience, San Diego, Calif.) 750 ml flasks with 2 pg/mL anti-CD28 (CD28.2, eBioscience) and 300 U/mL of human IL-2 in AIM V medium (Invitrogen, Grand Island, N.Y.) supplemented with 5% heat inactivated sterile human serum (Valley Biomedical, Winchester, Va.). 293T-HEK phoenix amphotropic cells (Orbigen, Allele Biotechnology, San Diego, Calif.) were transfected with 50 μg 1^{st} or 2^{nd} gen cKIT ligand CIR retroviral plasmid using LipoD283 (SignaGen Laboratories, Rockville, Md.). Viral supernatant was harvested for transduction of NIH-3T3 PG13 retrovirus packaging cells (ATTC: CRL-10686) cells that had reached 80% confluence. PG13 cells were cultured at 37° C. and supernatant was harvested and filtered through 0.45 μm filters (Corning, Corning N.Y.) when cells reached 80% confluence. After 24-48 hours of culture, PBMC were seeded on retronectin-coated (20 μg/mL, Takara Bio, Otsu, Shiga, Japan) wells of a 6-well plate and were transduced with anti-KIT CIR vector as described in Emtage et al., Clin Cancer Res 14:8112-8122, 2008 to create designer T cells. Cells were transduced with supernatant containing either anti-KIT CIR vector (1^{st} gen) or anti-KIT CIR vector with additional CD28 moiety (2^{nd} gen). Transduced T cells were maintained in AIM V medium supplemented with 5% heat inactivated sterile human serum and 100 IU/ml IL-2. Expression of KIT-specific CIR on designer T cells was evaluated by flow cytometric analysis of staining with Reprokine anti-SCF mAb (Fx2) conjugated to allophycocyanin-XL (Chromaprobe, Maryland Hts, Mo.). Cell were also stained with antibodies against human CD3 (Sk7), CD4 (RPA-T4), CD8 (SKI), CD62L, CD45RO, CD197 (CCR7, 150503), and CD25 (M-A251), which were conjugated to FITC, PE, PerCP, allophycocyanin, allophycocynanin-Cy7, or Pe-Cy7 (BD Biosciences, Franklin Lakes N.J.). For FoxP3 intracellular staining, samples were fixed, permeabilized, and stained with FoxP3 conjugated to PE as per manufacturer's protocol (BD).

### Cell Proliferation Assay

Flow cytometry-based division assays were performed to analyze the proliferation of 1^{st} and 2^{nd} gen designer T cells in response to stimulation by KIT+ human GIST cell lines. Designer T cells were labeled with 1 μM carboxyfluorescein diacetate succinimidyl ester (CFSE, Invitrogen) and were added at a 4:1 ratio with KIT+ GIST 882 and GIST 48 cells in a 96-well round-bottom plate, with 1×10⁵ dTc added per well. GIST 48b, which lacks KIT surface expression, was used a negative control. Tumor cells were irradiated at 5000 rad. Co-culture was incubated for 5 days, at which point supernatant was isolated and cells were analyzed by flow cytometry. Supernatant was analyzed by cytometric bead array for IFN-γ levels (BD Biosciences). Cytokine production results were also quantified by human IFN-γ ELISA assay for confirmation (Biolegend).

### Cytotoxicity Assays

1^{st} and 2^{nd} gen designer T cells were cultured with KIT+GIST 882 or GIST 48 cells in order to evaluate their cytotoxic ability in an LDH assay (Roche, Indianapolic, Ind.) performed according to the manufacturer's protocol. Tumor cells were irradiated at 5000 G for 50 minutes. Cytotoxic ability was evaluated for 1^{st} gen designer T cells, 2^{nd} gen designer T cells, and untransduced human T cells, which were added at various effector-to-target ratios. Cytotoxicity results from the LDH assay were further confirmed by flow cytometric analysis of tumor cell death. GIST cells were irradiated as previously described and labeled with CFSE while designer T cells were unstained. Loss of CFSE+ cells was analyzed with flow cytometry and cytotoxicity was calculated using the following formula: mean fluorescence index (MFI) 1^{st} or 2^{nd} gen designer T cells divided by MFI untransduced T cells. Tumor cells were stained with Annexin-V (BD Biosciences).

### In Vivo Tumor Studies

Six-week-old male immunodeficient mice (NU/J) were purchased from Jackson Laboratories (Bar Harbor, Me.) and experiments were conducted in compliance with the guidelines of the Roger Williams Medical Center Institutional Animal Care and Use Committee. The GIST cell lines were maintained at 37° C. in IMDM with 1% I-glutamine (Invitrogen) supplemented with 15% FBS, and 1%

Penicillin/Streptomycin/Amphomycin (Cellgro). Subcutaneous flank injections of 3×10⁷ KIT+ GIST 882 cells in 200 μl sterile PBS were administered bilaterally. Designer T cells or untransduced human T cells were injected (1×10⁷ in 200 μl PBS) via tail vein. For experimental groups with IL-2, Alzet 7-day micro-osmotic pumps (Durcet, Cupertino Calif.) were filled with IL-2 according to the manufacturer's protocol and implanted subcutaneously. Pumps were set to deliver at a rate of 10,000 IU/h (550 pg/h). Tumors were measured in two dimensions with calipers, and measurements were obtained daily from the time of T cell injection until the conclusion of the study. The average of right and left flank tumors was used for each mouse, and measurements were normalized to initial tumor size. After sacrifice, tumors were excised and sent to the University of Massachusetts, Worchester Medical Center Experimental Pathology Service Core, for histological sectioning and staining. Sections were stained for routine H&E and anti-CD3 immunohistochemistry. Slides were analyzed at the Pathology department at Roger Williams Medical Center, and photographs were taken under 10× and 40× magnification.

### Statistics

Statistics were calculated using GraphPad Prism V5.00 for Windows (GraphPad Software, San Diego, Calif.). Statistical significance for proliferation and cytotoxicity assays was determined using the two-tailed Student t test, and values with p≦0.05 were classified statistically significant. Tumor size median values are presented and logistic regression was used to compare growth curve slope and elevation among groups. Cell proliferation analysis with calculation of division peaks was performed using FlowJo software (Treestar, Ashland, Oreg.).

### Example 1

### Engineering of Anti-KIT Chimeric Immune Receptors and Production of Designer T Cells

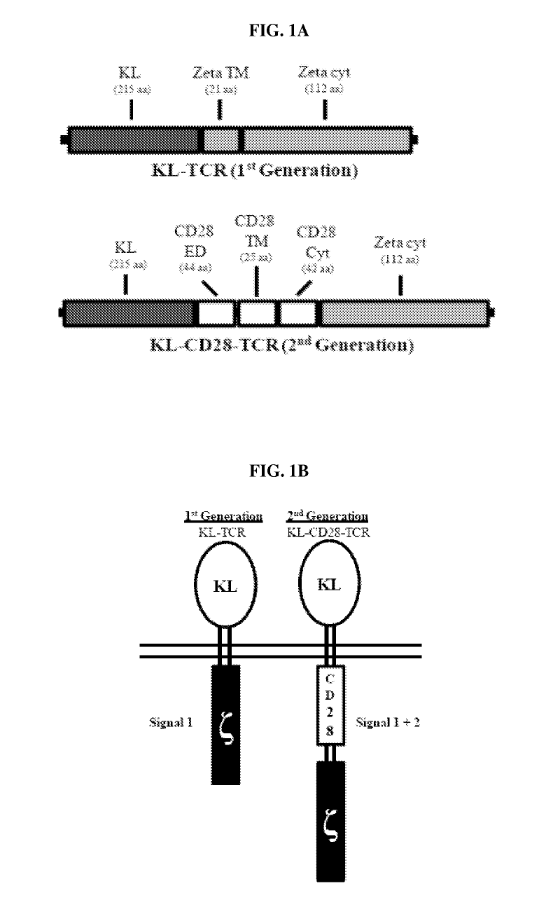

The aim of this study was to construct and test the function of KIT-specific CIR expressed by human peripheral blood T cells for pre-clinical development. The anti-KIT CIR construct was based on a pre-existing anti-CEA format described in Emtage et al., Clin Cancer Res 14:8112-8122, 2008. The anti-CEA sFv fragment was replaced with the extracellular domain of KL. 1^{st} and 2^{nd} gen constructs were prepared (FIG. 1A). The 2^{nd} gen construct contains CD28 to provide co-stimulation. The KL component is expressed on the extracellular aspect of the CIR to enable interaction with KIT on the surface of target tumor cells (FIG. 1B). The constructs were confirmed by direct DNA sequencing prior to transduction of activated lymphocytes.

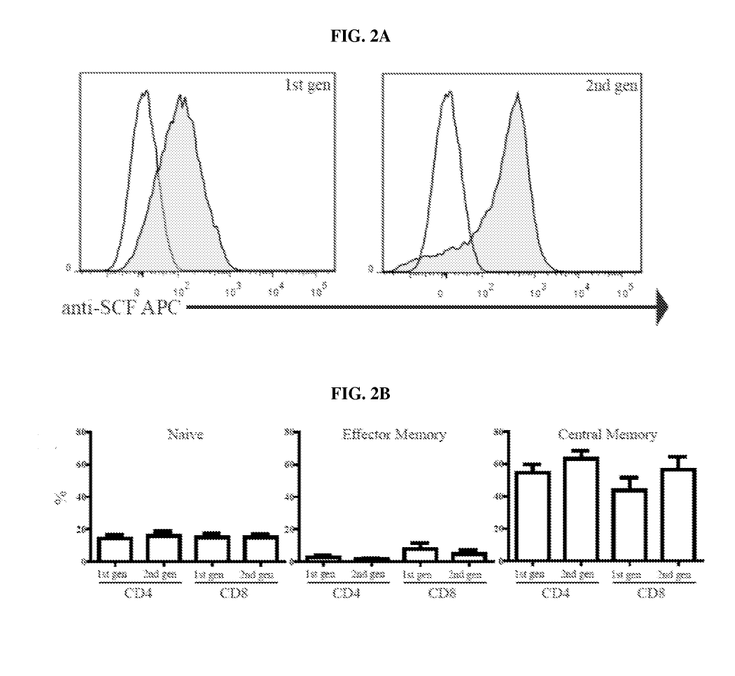

Following activation and transduction, CIR expression was confirmed by flow cytometry with an anti-KL antibody. Retroviral Transduction of activated murine splenocytes, which was used as a preliminary assessment, resulted in 1^{st} and 2^{nd} gen CIR expression rates of 27% (range, 16-41). Following optimization of the protocol, transduction of activated human PBMC (FIG. 2A) yielded mean transduction rates of 50% (range, 33-74) and 42% (range, 24-62) for 1^{st} and 2^{nd} gen human designer T cells respectively, with no significant difference between the two CIR versions (p=0.67). After stimulation of PBMC with anti-CD3, anti-CD28, and IL2, >70% of the cells were CD3+ and a central memory phenotype (CD45RO+CD62L+CCR7+) predominated (FIG. 2B). Fewer than 30% of cells had a naïve (CD45RO−CD62L+) or effector memory (CD45RO+CD62L+CCR7−) phenotype, and less than 10% of transduced T cells from both generations had a regulatory T cell phenotype (CD25+FoxP3+) with no differences between the groups. For 1^{st} gen dTc, 33.4% of T cells were CD4+CD8− and 52.7% were CD8+CD4−, while the corresponding values for 2^{nd} gen dTc were 35.5% and 52.6%. The CD4:CD8 ratio did not change after transduction or exposure to KIT+ tumor. For all subsequent experiments, designer T cells were used in bulk, without fractionating by CD4 or CD8 expression, in keeping with current clinical practice.

### Example 2

### Proliferation of Anti-KIT Designer T Cells in the Presence of KIT+ Tumor Cells

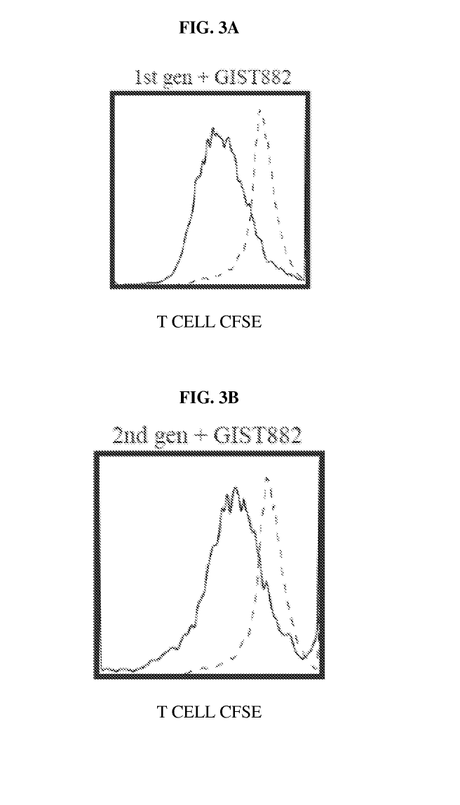

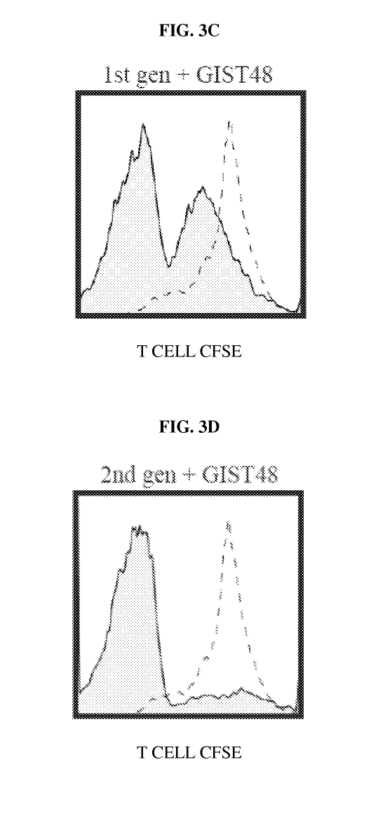

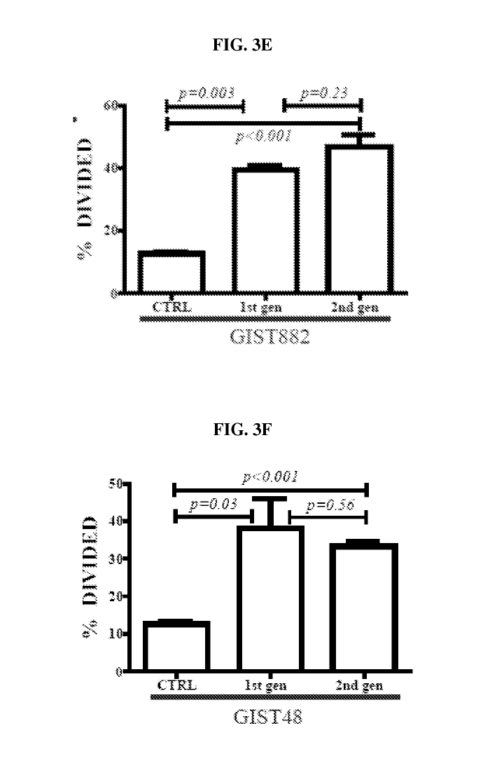

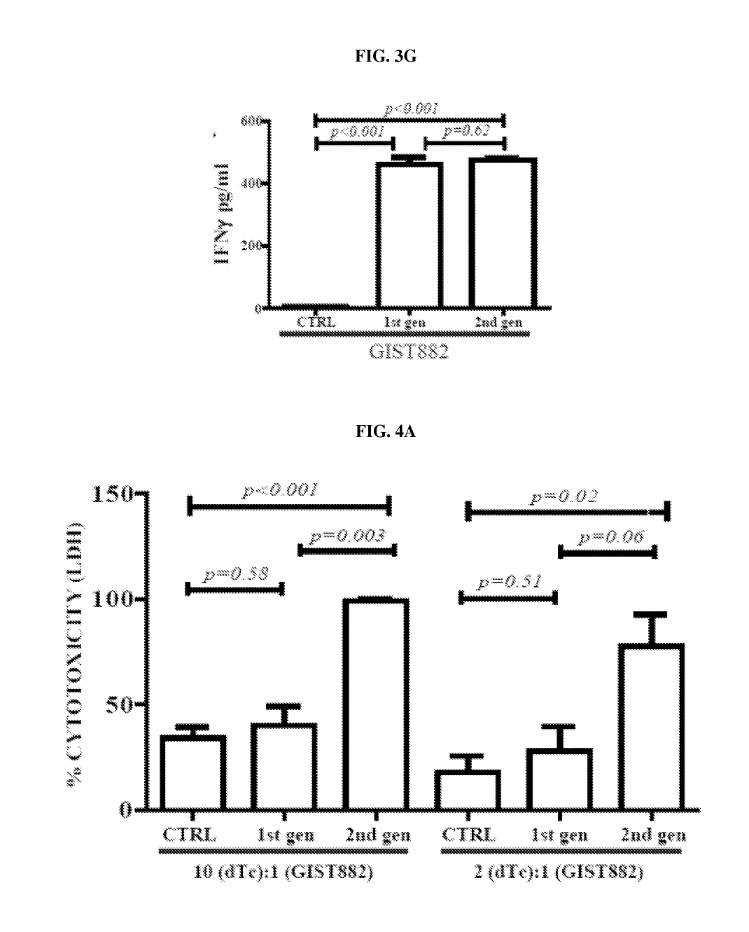

To test the proliferative capacity of human T cells expressing anti-KIT CIR, we cultured the dTc in the presence of two human KIT+GIST cell lines, GIST882 and GIST48. In the presence of GIST882 and GIST48, dTc expressing either the 1^{st} or 2^{nd} gen anti-KIT CIR proliferated to a greater extent when compared to untransduced T cells (CTRL) as determined by CFSE dilution (FIG. 3A). When cultured with GIST882, 39% of the 1^{st} gen and 47% of the 2^{nd} gen dTc divided (p<0.001 compared to CTRL), with no significant difference between the two CIR formats (p=0.23, FIG. 3B). Likewise, in the presence of imatinib resistant GIST48 cells, 33-38% of the dTc divided after 3 days in culture which was significantly higher than CTRL cells (130.03 compared to CTRL), with no significant difference between the two CIR formats (p=0.56, FIG. 3C). The requirement of KIT+ tumor cells for dTc proliferation was confirmed by the minimal proliferation that resulted when culturing dTc in the presence of KIT-GIST 48B cells as shown, and CIR-activated T cells did not proliferate in the presence of KIT+ tumor. IFNγ production confirmed dTc activation by KIT+ tumor and was found to be in the range of 462-475 pg/ml, while production by CIR-T cells was negligible (p<0.001, FIG. 3D). Co-culture of anti-KIT dTc with KIT-control tumor cells did not result in significant IFNγ production.

### Example 3

### Lysis of KIT+ Tumor Cells by Anti-KIT Designer T Cells

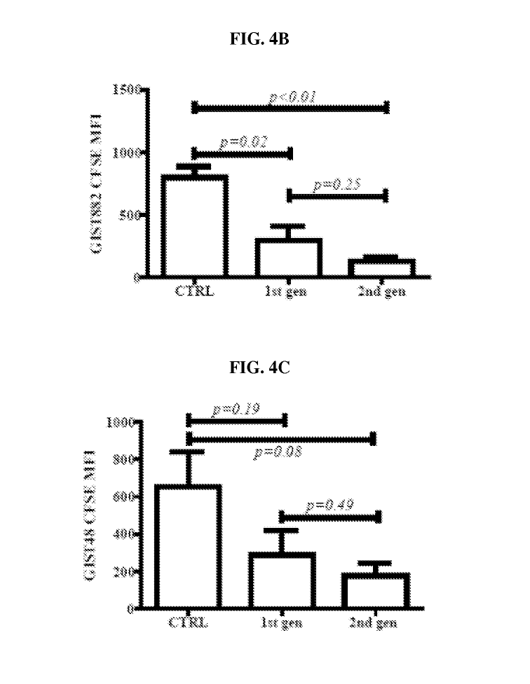

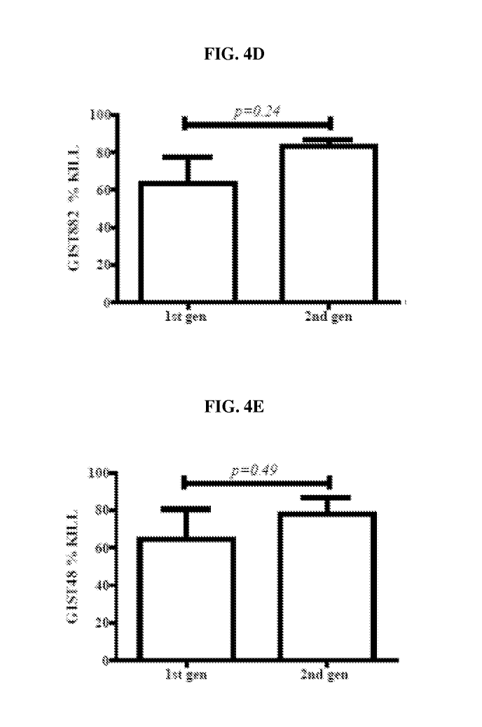

The hallmark of effective adoptive cellular immunotherapy is the ability of the product to lyse tumor cells in a specific fashion. To this end, in vitro assays were performed to determine if designer T cells expressing anti-KIT CIR were able to destroy GIST cells. It was demonstrated that 2^{nd} gen designer T cells effectively lysed KIT+ tumor and were more effective than the 1^{st} gen format by LDH release (FIG. 4A). To confirm these findings, CFSE-labeled irradiated tumor cells were mixed with unlabeled designer T cells. Tumor cell loss was measured using by quantifying the decrease in CFSE fluorescence from remaining live cells. When compared to CTRL cells, 1^{st} gen and 2^{nd} gen designer T cells mediated significant decreases in the level of CFSE fluorescence and hence number of live tumor cells (FIGS. 4B-4C). Having demonstrated that the anti-KIT designer T cells were stimulated to divide in vitro in response to KIT+ tumor and lyse KIT+ targets, in vivo efficacy was measured next.

### Example 4

### In Vivo Assessment of Anti-KIT Designer T Cells

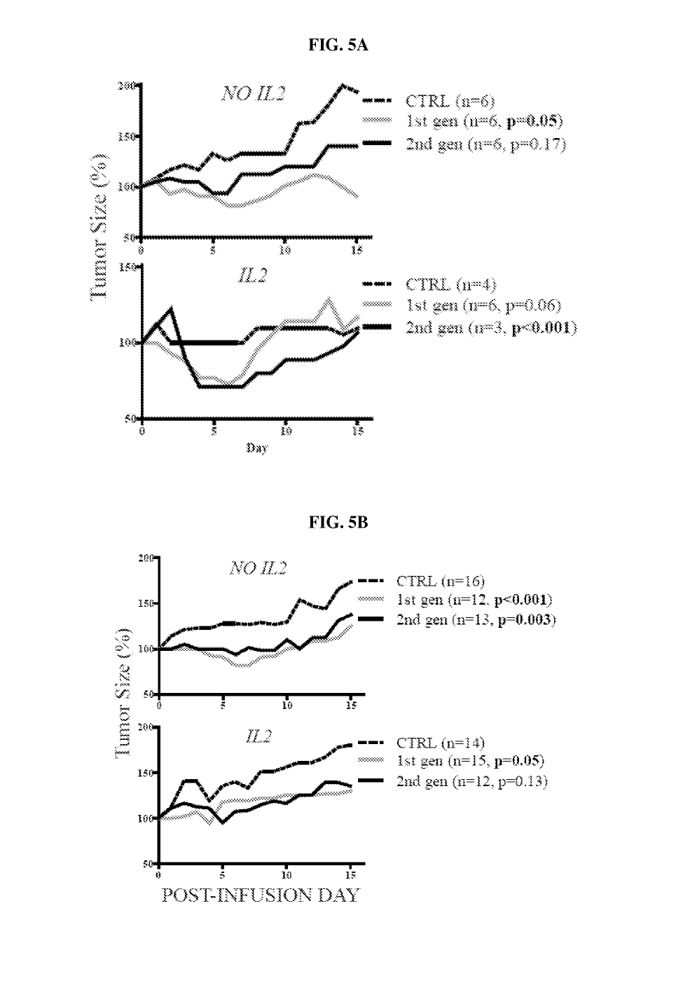

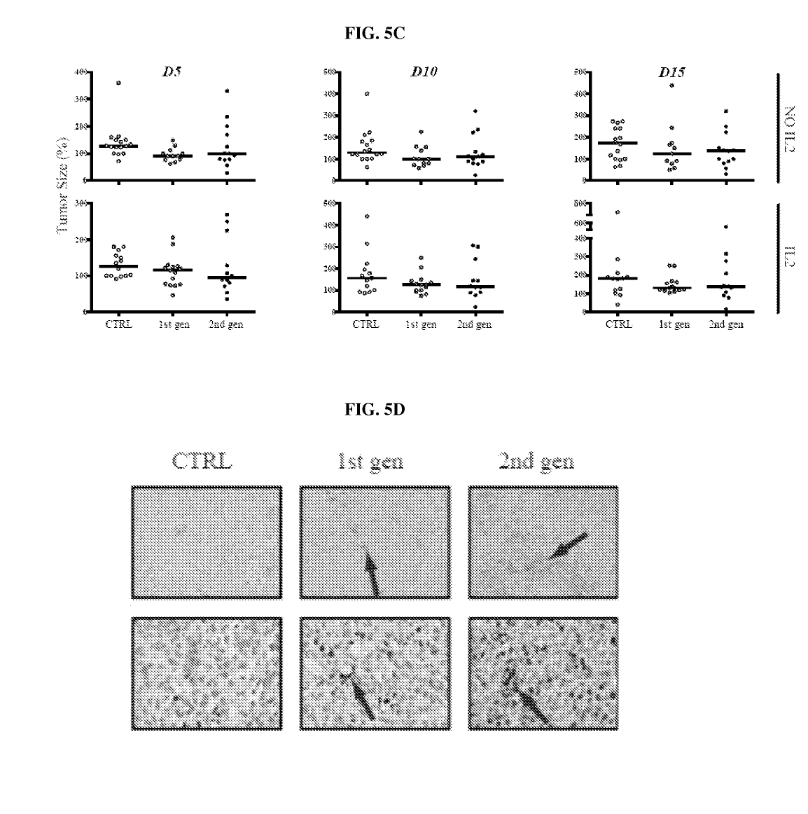

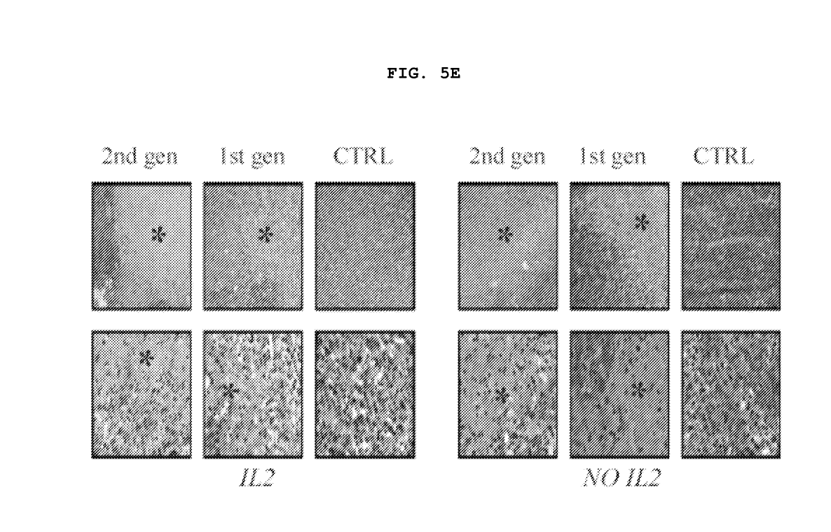

To determine the ability of anti-KIT designer T cells to traffic to, infiltrate, and limit growth of established tumor, a subcutaneous xenograft model was utilized. Human KIT+GIST cells were injected subcutaneously into immunodeficient mice that were treated with tail vein injections of 1^{st} gen, 2^{nd} gen, or unmodified anti-KIT designer T cells seven days later. Tumor measurements were performed in two dimensions (mm²) and are expressed as median percentage change relative to the tumor size on initial day of treatment. IL2 therapy was given along with designer T cells for some groups. Significant reductions in tumor growth were mediated by 1^{st} gen designer T cells without IL2 (p=0.05) and 2^{nd} gen designer T cells (p<0.001) with IL2 (FIG. 5A). When all data were pooled, both 1^{st} and 2^{nd} gen designer T cells had a significant impact on tumor growth in the absence of IL2 therapy. With IL2 support, 1^{st} gen designer T cells had a significant effect (p=0.05) while 2^{nd} gen designer T cells (p=0.13) demonstrated a favorable trend (FIG. 5B 1^{st} gen dTc may be more reliant on IL2 than 2^{nd} gen dTc because the presence of the co-stimulatory signal through the CD28 portion of the construct may reduce the dependence of 2^{nd} gen dTc on cytokines such as IL2. Data is further represented as tumor growth for each individual sample to demonstrate the range of values (FIG. 5C). Following sacrifice, we harvested the tumors and confirmed the presence of adoptively transferred dTc and necrosis in animals treated with 1^{st} or 2^{nd} gen dTc (FIGS. 5D and 5E).

### Other Embodiments

Various modifications and variations of the described methods and compositions of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

All publications mentioned in this specification are herein incorporated by reference to the same extent as if each independent publication was specifically and individually incorporated by reference.

What is claimed is:

1. A nucleic acid construct encoding a chimeric immune receptor (CIR) protein comprising
a. an extracellular domain of KIT-ligand (KL), or a fragment thereof,
b. a transmembrane domain, and
c. a cytoplasmic domain,
wherein said transmembrane domain comprises a domain of the CD3 zeta chain, or a fragment thereof, and said cytoplasmic domain comprises a domain of the CD3 zeta chain, or a fragment thereof.

2. A nucleic acid construct encoding a CIR protein comprising
a. an extracellular domain of KIT-ligand, or a fragment thereof,
b. a transmembrane domain, and
c. a cytoplasmic domain,
wherein said transmembrane domain comprises a domain of CD28, or a fragment thereof, and said cytoplasmic domain comprises a domain of the CD3 zeta chain and a domain of CD28, or fragments thereof.

3. The nucleic acid construct of claim 2, wherein said extracellular domain further comprises a domain of CD28, or a fragment thereof.

4. The nucleic acid construct of claim 1, wherein said CIR protein, when expressed in a T cell, is capable of activating said T cell in the presence of a tyrosine-protein kinase KIT+ (KIT+) tumor cell.

5. The nucleic acid construct of claim 1, wherein said extracellular domain of said CIR protein is capable of interacting with KIT on the surface of a tumor cell when expressed in a T cell.

6. A vector comprising the nucleic acid construct of claim 1.

7. A host cell comprising the nucleic acid construct of claim 1.

8. The host cell of claim 7, wherein said host cell is selected from the group consisting of a T cell, a hematopoietic stem cell, a natural killer cell, a natural killer T cell, a B cell, and a cell of monocytic lineage.

9. A method of destroying a KIT+ cell, said method comprising administering a composition comprising the nucleic acid construct of claim 1.

10. A method of destroying a KIT+ cell, said method comprising contacting said KIT+ cell with a composition comprising the host cell of claim 7.

11. The method of claim 10, further comprising administering to said KIT+ cell a second agent.

12. The method of claim 11, wherein said second agent is a tyrosine-kinase inhibitor.

13. A method of treating a subject with a KIT+ associated disease, said method comprising administering a composition comprising the nucleic acid construct of claim 1.

14. A method of treating a subject with a KIT+ associated disease, said method comprising administering a composition comprising the host cell of claim 7.

15. The method of claim 14, wherein said KIT+ associated disease is characterized by the presence of KIT+ tumor cells.

16. The method of claim 15, wherein said KIT+ associated disease is selected from the group consisting of: gastrointestinal stromal tumor, acute myelogenous leukemia, small-cell lung carcinoma, ovarian carcinoma, breast carcinoma, melanoma, neuroblastoma, and soft-tissue sarcomas of neuroectodermal origin.

17. The method of claim 16, wherein said KIT+ associated disease is gastrointestinal stromal tumor (GIST).

18. The method of claim 17, wherein said GIST is resistant to imatinib mesylate.

19. The method of claim 14, wherein said host cell is autologous to said subject.

20. The method of claim 14, wherein said host cell is not autologous to said subject.

21. The method of claim 14, further comprising administering a second agent.

22.-23. (canceled)


SEQUENCE LISTING (6 sequences)

SEQ ID NO: 1 - protein, 273 aa, homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 2 - DNA, 45 bp, homo sapiens
gattccaggaattgatttccccatggcaaagaagacacaaacttg

SEQ ID NO: 3 - DNA, 46 bp, homo sapiens
ctaagctctagccaattgaattggatccgtgtaggctggagtctcc

SEQ ID NO: 4 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 5 - protein, 163 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 10 aa, Homo sapiens
EQKLISEEDL